(19) United States
(12) Patent Application Publication
(10) Pub. No.: US 2008/0215017 A1
(43) Pub. Date: Sep. 4, 2008

(54) METHOD OF MAKING COBALT-BASED ALLOY TUBES HAVING ENHANCED MECHANICAL PERFORMANCE CHARACTERISTICS AND A TUBE FORMED BY THE METHOD

(76) Inventors: Vladimir Gergely, Oranmore (IE); Enda Keehan, Ennis (IE); Conan Cavanagh, Claregalway (IE)

Correspondence Address: SCHIFF HARDIN, LLP;PATENT DEPARTMENT, 6600 SEARS TOWER, CHICAGO, IL, 60606-6473, US

(21) Appl. No.: 11/971,494

(22) Filed: Jan. 9, 2008

Related U.S. Application Data
(60) Provisional application No. 60/888,661, filed on Feb. 7, 2007.

(51) Int. Cl. A61M 25/06; C22F 1/00

(52) U.S. Cl. 604/272; 604/523; 148/519

(57) ABSTRACT

A method for the manufacture of cobalt-based tubes is provided, wherein cobalt alloy strips are formed into tubes, and the drawn tubes are subjected to hardening and/or stress relieving by heat treatment. A synergistic effect of the material composition, the tube drawing and the heat-treatment process results in production of tubes with significantly enhanced mechanical performance attributes.

### CROSS-REFERENCE TO RELATED APPLICATION

The present application claims the benefit of the U.S. Provisional Patent Application Ser. No. 60/888,661, filed Feb. 7, 2007, which is incorporated herein by reference.

### BACKGROUND OF THE INVENTION

1. Field of the Invention

The present invention relates generally to a catheter or needle, and more particularly to a tube, also termed a hypotube in some embodiments, for intravascular, endoscopic, intramuscular or transdermal use in the body, for example to deliver treatments, devices, to sample cells and the like.

2. Description of the Related Art

A hypotube is the tube portion of a catheter or needle used to deliver stents, angioplasty devices or other instruments into the body or for aspiration, insufflation, injection or biopsy sampling. In a typical use of a catheter hypotube device, the elongated hypotube is provided with a treatment device at its end. For example, it can be introduced into the femoral artery at the leg and is fed along the arterial system to the heart, where it is guided into selected arteries so that the hypotube device may deliver the treatment device to the desired location.

The performance characteristics of the hypotube shaft have established the hypotube as the device shaft of choice for PTA (Percutaneous Transluminal Angioplasty) applications. Recognizing its superior performance benefits, leading companies are adopting hypotube-based device shafts in new application areas such as neurology, peripheral vascular interventions and catheter-based imaging.

A hypotube is a long shaft that often has micro-engineered features along its length. It is one of the components of minimally-invasive catheter systems. A hypotube is used for delivering balloons, stents and other devices into a human or animal anatomy. In needle applications it is used for aspiration, insufflation or biopsy sampling or for delivery of treatment. The hypotube enters the body and pushes the attached device along what may be a torturous path. This journey requires hypotubes to resist kinking as well as to possess other attributes known as push, track, torque and shape set resilience.

The most common materials currently used for the manufacture of metallic hypotubes are AISI 300-series austenitic stainless steels. While hypotubes manufactured from these steels can tolerate a reasonably high displacement before they fail locally under axial compression, or kink, the actual column strength of these hypotubes is relatively low. On the other hand, hypotubes manufactured from 17-7PH stainless steel can exhibit higher column strength than 300-series stainless steels but undergo less displacement before they kink. It is a common issue encountered by hypotube designers that improvement of one of the hypotube performance attributes comes at the expense of another one.

### SUMMARY OF THE INVENTION

The present invention provides a catheter or needle hypotube exhibiting a superior combination of column strength, kink resistance and shape set resilience. In particular, the hypotube is formed according to a method wherein a cobalt-based alloy strip is shaped, welded, drawn and heat-treated to an elongated thin tube having a desired column strength and kink resistance. In a preferred embodiment, the invention provides a hypotube and method for its manufacture from a cobalt-based alloy where a significant improvement of column strength and kink resistance is obtained.

In a further development, the Co-based alloy and the alloy hypotubes/tubes have a great applicability in medical needles. These needles have endoscopic, intramuscular or transdermal use to deliver treatments, devices or to sample cells in the body. In the technical field relating to needles, the metal tubes that make up the needles are generally referred to as “tubes”.

The preferred cobalt-based alloy for use in the present invention is specified by ASTM (American Society for Testing and Materials Standards) F1058 and ISO (International Organization for Standardization) 5832-7 and also known as Conichrome®, Phynox™ or Elgiloy® (See Table 1 for the chemical composition of these alloys). The Elgiloy® alloy was developed and patented originally by Batelle Laboratories for watch springs in 1950. The drawing process of tubes from the cobalt-based alloy is similar in some respects to the drawing from AISI 300-series austenitic stainless steels. The tubes obtain the required properties from a combination of cold work and thermal processing of the alloy.

In order to tailor the cobalt-based alloy for the subsequent heat-treatment, it is important to design the tube drawing process such that the tensile properties of the tubes are within the range of the tensile properties exhibited by half- to full-hard hypotubes drawn from the AISI 300-series austenitic stainless steels.

The heat treatment of the as-drawn cobalt-based alloy (Phynox™) tubes in the present invention comprises heating the tubes under vacuum and/or inert atmosphere at a temperature in the temperature range from about 100° C. to about 475° C. over a time period from about 5 minutes to about 10 hours. This heat treatment range not only notably increases the tube's critical buckling force but also delivers a kink resistance that is comparable to or better than the kink resistance of 304 or 17-7PH steel-based hypotubes.

### BRIEF DESCRIPTION OF THE DRAWINGS

The foregoing and other advantages of the invention will be appreciated more fully from the following description thereof, with reference to the accompanying drawing wherein:

FIG. 1a is a schematic illustration showing a beginning of a column strength test on a tube;

FIG. 1b is a schematic illustration of a further stage in the column strength test of FIG. 1a;

FIG. 1c is a schematic illustration of yet a further stage in the column strength test of FIG. 1a;

FIG. 1d is graph of a typical force-displacement curve recorded during a column strength test on a tube;

FIG. 2 is a plot showing an example of force/displacement curves of as-drawn 304L steel and as-drawn and heat-treated Phynox alloy hypotubes tested according to the column strength test shown in FIGS. 1a-1c;

FIG. 3 is a graph providing a diagrammatic example of the critical buckling force and the displacement at kink values for as-drawn 304L steel hypotubes, as-drawn cobalt alloy hypotubes, heat-treated 17-7PH steel hypotubes and heat-treated cobalt alloy-based hypotubes (the composition of which are set forth in Table 1). The tubes were tested according to the column strength test shown in FIGS. 1a-1c;

### DETAILED DESCRIPTION OF THE PREFERRED EMBODIMENTS

The present invention provides a method and product produced by the method, or process, for producing a tube having an improved kink resistance, columnar strength and shape set resilience. According to a preferred embodiment, the hypotubes are manufactured from a cobalt-based alloy specified by the standards ASTM F1058 and ISO 5832-7. The typical composition range of the cobalt-based alloy is provided in the examples set forth in Table 1. The composition of the alloy is given in weight percent in the table.

Forming, welding and drawing hypotubes from the cobalt-based alloy strip is carried out. These steps are similar to those used by those skilled in the art of drawing hypotubes from AISI 300-series austenitic stainless steels. An example of such steps include forming the strip into a tube shape by roll-forming, seam welding the seam to form the tube, and drawing the welded tube to form an elongated tube. The drawing process may be performed in several drawing steps. This may involve both plug drawing and sinking. A heating step may be provided between each drawing step. The drawing steps may be performed as cold work. In order to condition the cobalt-based alloy for the subsequent heat treatment, the amount of cold-work during the drawing process should be selected in such a way that the tensile property range of the as-drawn hypotubes manufactured from the cobalt-based alloy falls into the tensile property range typically exhibited by half- to full-hard hypotubes drawn from AISI 300-series austenitic stainless steels.

The heat treatment of the cobalt-based alloy hypotubes typically comprises heating the tubes under vacuum and/or under an inert atmosphere at a temperature in the temperature range from about 100° C. to about 475° C. from about 5 min to about 10 hours. The temperature and duration of heat-treatment are varied depending on the exact composition of the cobalt-based alloy, the amount of cold work the material is subjected to during the tube drawing process and the targeted combination of mechanical performance characteristics.

It is demonstrated by the following examples that the present combination of material composition, tube drawing process, and heat-treatment process provides significant mechanical performance advantages over AISI 304L and 17-7PH steel-based hypotubes. In the examples given, the as-drawn cobalt-based alloy hypotubes are preferably subjected to heat-treatment at temperatures from about 100° C. to about 475° C.

Table 2 gives some examples of the evaluated tubes, their material type, conditions (as-drawn or heat-treated), tensile properties (UTS: ultimate tensile test, YS: yield strength at 0.002 offset strain and Elongation over a gauge length of 50 mm) and shape-set resilience properties. The tube dimensions (OD: outer diameter and ID: inner diameter) are listed in Table 3.

Mechanical performance characteristics of the hypotubes listed in Table 2 were evaluated using the following test methods:

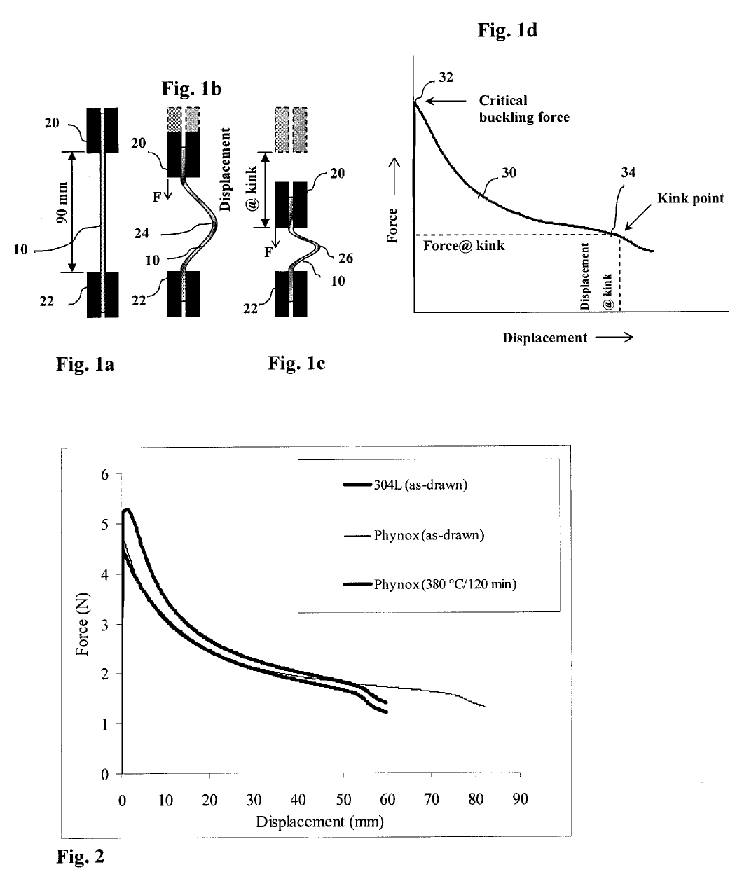

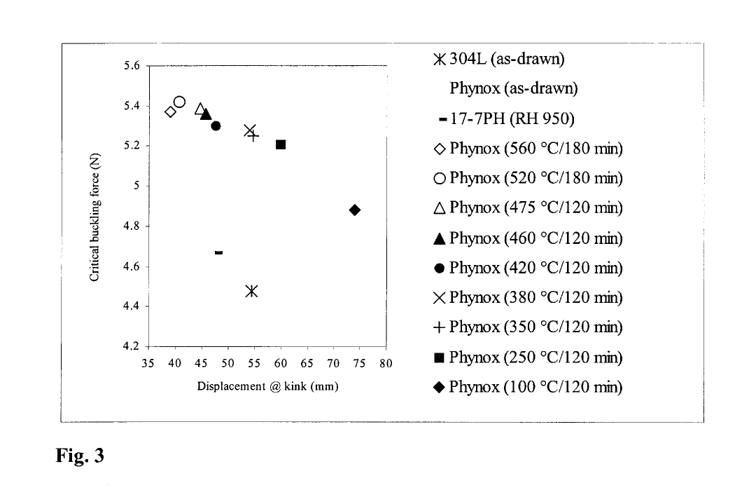

Test 1: Column strength test. This test is conducted by applying an axial compression load to a tube 10 as shown in FIG. 1a. In the test, the tube 10 is gripped at two locations along its length by grippers 20 and 22. The grippers 20 and 22 grasp the tube 10 tightly enough to avoid slipping on the tube. In one embodiment, the start distance between the grippers 20 and 22 is 90 mm. As shown in FIG. 1b, a force as indicated by arrow F is applied to move the gripper 20 towards the gripper 22. As the force F is applied, the gripper 20 initially moves only negligibly. This is as a result of the columnar strength of the tube 10. As greater force is applied, the tube 10 begins to bend outwards at the unsupported middle section 24. To bend outwardly as indicated in FIG. 1b, the tube actually bends in three different bend directions with bends in a first direction nearer the grippers 20 and 22, respectively, and a reverse bend at the mid portion 24. In FIG. 1c, the tube 10 is bent to a relatively extreme deformation and a kink (wherein one side of the tube wall collapses) has occurred in the tube, typically in the tube mid-section 26. Once the tube 10 kinks, the tube 10 offers markedly lower resistance to further bending.

A graph of the typical load/displacement curve for a column strength test is shown in FIG. 1d. A curve 30 plots the positions of the critical buckling force at 32 and the kink point 34 along its plot. These are not only locations of changes in the shape of the tube but are also locations of rapid changes in the curve of the force plot.

Test 2: Shape set resilience test. This test involves storing a tube in a coiled shape for a period of time and then measuring the curvature retained by the tube after the tube is permitted to return to its relaxed shape. In one example, a tube 10 is stored in a 152 mm diameter coil for twelve hours. After removal of the 1 meter long tube from the storage case coil, the shape set value, h, is defined as a perpendicular distance between a ruler (that is touching both tube outer ends) and a highest point of the tube arc.

The foregoing tests were applied to batches of hypotubes prepared according to the heat treatment characteristics listed in Table 2. Also shown in Table 2 are the tensile properties (UTS: ultimate tensile test, YS: yield strength at 0.002 offset strain and Elongation over a gauge length of 50 mm) and the shape set resilience test results. The results for each batch were summarized or averaged over the plurality of hypotubes in each batch and are indicated as a single entry for each table entry.

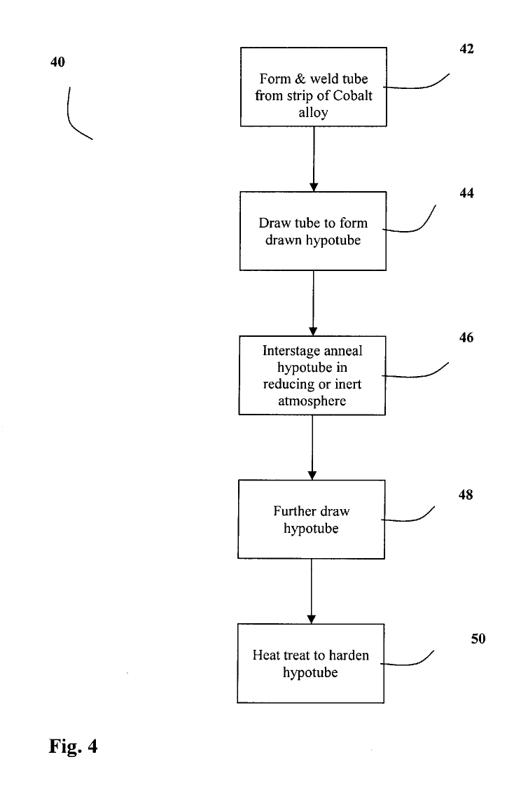

A comparison between the column strength test (Test 1) results from the cobalt-based alloy, the AISI 304L and the 17-7PH (RH950) steels is presented in FIG. 3. It is observed that all heat-treated cobalt-based alloy hypotubes exhibit higher critical buckling force than 304L and 17-7PH (RH950) steel hypotubes.

A comparison of results from the 17-7PH (RH950) hypotubes and the cobalt-based alloy-based hypotubes heat treated at low temperatures in FIG. 3 also shows that the cobalt-based alloy-based hypotubes exhibit significantly higher displacement at kink than the 17-7PH (RH950) hypotubes.

It is also clearly observed from the graph in FIG. 3 that heat-treatments of the cobalt-based hypotubes at progressively higher temperatures result in a significant increase in critical buckling force.

From these examples, the Phynox hypotubes that have been heat-treated within the temperature range 100-380° C./120 min offers the best combination of both critical buckling force, displacement at kink and shape set resilience.

Comparing the as-drawn Phynox and the Phynox heat-treated at the lowest temperature (100° C./120 min) from the given examples, it is clear that the heat treatment of the cobalt-based alloy even at such a low temperature is beneficial since the hypotubes can withstand a notably higher buckling force with minimum loss in displacement at the kink.

The Phynox hypotubes which have heat treated at a temperature of 520° and 560° C. can only tolerate a relatively small displacement before they kink. These small displacement values are unlikely to be acceptable in catheter-based applications.

The benefit of the heat treatment is also reflected on the shape set resilience values h, as tested in Test 2, with the results listed in Table 2. Hypotubes with smaller h values have a better shape set resilience. It can be seen that the shape set resilience of all heat-treated Phynox hypotubes are better than those of the as-drawn Phynox, 304L and 17-7PH (RH 950) hypotubes. This means that the heat treated Phynox hypotubes are much less likely to acquire a permanent shape set after being stored in a coiled state, during handling, etc.

In FIG. 4, a method 40 according to a preferred embodiment of the present invention provides for the manufacture of cobalt alloy hypotubes exhibiting enhanced mechanical performance characteristics, particularly a favorable combination of columnar strength, kink resistance and shape set resilience. The methods of a preferred embodiment include the following steps. The tube is formed in step 42 where a strip is shaped and welded, such as a strip of cobalt alloy having a composition corresponding to 40 cobalt-20chromium-16iron-15nickel-7molybdenum alloy (UNS R30003 and UNS 30008). The tube is drawn to form a drawn hypotube, according to step 44. The drawn hypotube is interstage annealed, for example in an atmosphere of an inert gas or in a reducing atmosphere, as shown at step 46. This can provide a hypotube according to some embodiments, but more commonly at least one or more further drawing and annealing steps are performed. Thus, a further drawing step 48 is performed on the hypotube, followed by a hardening heat treatment 50.

In one example, the method for the manufacture of cobalt-based tubes exhibiting enhanced mechanical performance characteristics includes the steps of: (a) drawing hypotubes from a cobalt-based alloy as described in the preceding paragraph; and (b) heating the as-drawn hypotubes under a vacuum and/or an inert atmosphere in the temperature range from about 100° C. to about 475° C. for about from 5 minutes to about 10 hours.

In one embodiment, the present method provides that the composition of the cobalt-based alloy used for the hypotube is within the composition range of the commercially available alloys sold under the trade names Conichrome®, Phynox™ or Elgiloy®. For example, commercially available the Phynox alloy has an approximate composition by weight percent of: Co 39-42 wt. %, Cr 18-21 wt. %, Ni 15-18 wt. %, Mo 6-8 wt. % and Fe balance as the major components. In a specific example, the alloy composition is 40% Co, 20% Cr, 16% Ni and 7% Mo and Fe balance as major components. Minor components may be provided in addition to the major components, for example, another alloy composition is 40% Co, 20% Cr, 16% Ni, 15% Fe, 7% Mo, 2% Mn, 0.4% Si and 0.0037% C. Other minor components and trace elements may be used in the alloy, including but not limited to P, S and Be. Further examples of alloys that may be used in the present hypotube are set forth in Table 1. Other ranges of constituents are also possible within the scope of this invention.

In a further development, the method provides that the hypotubes are manufactured from a strip from the foregoing cobalt-based alloy.

According to preferred embodiments, the method provides that the ultimate tensile strength of the as-drawn hypotubes from the cobalt-based alloy is within the ultimate tensile strength range typically exhibited by half- to full-hard hypotubes manufactured from AISI 300-series austenitic stainless steels.

Preferably, the method provides the heat-treatment temperature range of the as-drawn hypotubes from the cobalt-based alloy at a temperature from about 100° C. to about 380° C. for approximately 1 to 3 hours, and preferably approximately 2 hours.

Thus, there is provided to a catheter or needle, and more particularly to a tube, also termed a hypotube, for intravascular, endoscopic, intramuscular or transdermal use in the body, for example to delivery treatments, devices, to sample cells and the like.

Although other modifications and changes may be suggested by those skilled in the art, it is the intention of the inventors to embody within the patent warranted hereon all changes and modifications as reasonably and properly come within the scope of their contribution to the art.

TABLE 1
Chemical composition (weight %)

| | |
|---|---|
| Phynox | Elgiloy |

| | Conichrome* | min | max | min | max |
|---|---|---|---|---|---|
| Carbon | 0.061 | — | 0.15 | — | 0.15 |
| Manganese | 1.97 | 1.0 | 2.0 | 1.5 | 2.5 |
| Silicon | 0.478 | — | 1.2 | — | 1.2 |
| Phosphorus | 0.005 | — | 0.015 | — | 0.015 |
| Sulphur | 0.0015 | — | 0.015 | — | 0.015 |
| Cobalt | 39.8 | 39.0 | 42.0 | 39.0 | 41.0 |
| Chromium | 19.9 | 18.5 | 21.5 | 19.0 | 21.0 |
| Nickel | 15.4 | 15.0 | 18.0 | 14.0 | 16.0 |
| Molybdenum | 7.1 | 6.5 | 7.5 | 6.0 | 8.0 |
| Beryllium | 0.0002 | — | 0.001 | — | 0.1 |
| Iron | bal. | bal. | bal. | bal. | bal. |

*Composition as specified by Fort Wayne Metals

TABLE 2

| | | | | | | Shape Set |
|---|---|---|---|---|---|---|
| | Heat-treated | | U.T.S. | Y.S. | Elongation | Resilience, |
| Material | (deg C.) | (min) | (kpsi) | (kpsi) | (%) | h [mm] |
| AISI 304L | As-Drawn | | 202 | 158 | 4.6 | 56 |
| 17-7PH | RH 950* | | 222 | 201 | 9.6 | 60 |
| Phynox | As-Drawn | | 211 | 139 | 11.3 | 86 |
| Phynox | 560 | 180 | 237 | 213 | 4.4 | 2 |
| Phynox | 520 | 180 | 234 | 211 | 4.6 | 1 |
| Phynox | 475 | 120 | 227 | 200 | 5.6 | 2 |
| Phynox | 460 | 120 | 225 | 195 | 5.8 | 2 |
| Phynox | 420 | 120 | 226 | 191 | 6.6 | 2 |
| Phynox | 380 | 120 | 220 | 181 | 8.0 | 2 |
| Phynox | 350 | 120 | 217 | 177 | 8.7 | 3 |
| Phynox | 250 | 120 | 216 | 169 | 8.9 | 4 |
| Phynox | 100 | 120 | 210 | 143 | 10.7 | 50 |

*RH 950 code refers to heat-treatment conditions described in open technical literature.

TABLE 3

| | Dimensions | | |
|---|---|---|---|
| Alloy | OD (mm) | ID (mm) | WT (mm) |
| AISI 304L | 0.63 | 0.46 | 0.09 |
| 17-7PH | 0.63 | 0.48 | 0.08 |
| Phynox | 0.63 | 0.47 | 0.08 |

We claim:

1. A tube for use in the body, comprising:
a hollow elongated tube formed of an alloy, the alloy having an approximate composition of major components of: Co 39-42 wt. %, Cr 18-21 wt. %, Ni 14-18 wt. %, Mo 6-8 wt. % and Fe balance, said hollow elongated tube having characteristics as a result of being cold-worked and heat treated in a temperature range of approximately 100 degrees C. to approximately 475 degrees C. for a period of from approximately 5 minutes to approximately 10 hours.

2. A tube as claimed in claim 1, wherein said hollow elongated tube is structured as a hypotube.

3. A tube as claimed in claim 2, wherein said hypotube is one of a catheter and a needle.

4. A tube as claimed in claim 1, wherein said hollow elongated tube has a characteristic as a result of being heat treated in a temperature range of approximately 100 degrees C. to approximately 380 degrees C.

5. A tube as claimed in claim 4, wherein said hollow elongated tube has a characteristic as a result of being heat treated for between 1 and 4 hours.

6. A tube as claimed in claim 4, wherein said hollow elongated tube has a characteristic as a result of being heat treated for approximately 2 hours.

7. A method for forming a tube for use in the body, comprising the steps of:
shaping a strip of cobalt-based alloy material into a tube-shaped strip, the cobalt-based alloy material having a composition consisting essentially of Co 39-42 wt. %, Cr 18-21 wt. %, Ni 14-18 wt. %, Mo 6-8 wt. % and Fe balance;
welding the tube-shaped strip to form a tube;
drawing the tube to form an elongated thin tube;
interstage annealing between drawing steps; and
heat treating the elongated thin tube in a temperature range of approximately 100 degrees C. to approximately 475 degrees C. for a period of from approximately 5 minutes to approximately 10 hours.

8. A method as claimed in claim 7, wherein said heat treating step is in a temperature range of approximately 100 degrees C. to approximately 380 degrees C. for approximately 2 hours.

9. A method for forming a tube for use in the body, comprising the steps of:
shaping a strip of cobalt-based alloy material into a tube-shaped strip, the cobalt-based alloy material having an approximate composition according to ISO 5832-7;
welding the tube-shaped strip to form a tube;
drawing the tube to form an elongated thin tube; and
heat treating the elongated thin tube in a temperature range of approximately 100 degrees C. to approximately 475 degrees C. for a period of from approximately 5 minutes to approximately 10 hours.

10. A method as claimed in claim 9, wherein the tube is formed into a hypotube.

11. A method as claimed in claim 10, wherein said hypotube is structured for one of a catheter and a needle.

12. A method for forming a tube for use in the body, comprising the steps of:
shaping a strip of cobalt-based alloy material into a tube-shaped strip, the cobalt-based alloy material having as major components: Co 39-42 wt. %, Cr 18-21 wt. %, Ni 14-18 wt. %, Mo 6-8 wt. % and Fe balance;
welding the tube-shaped strip to form a tube;
drawing the tube to form an elongated thin tube;
interstage annealing between drawing steps; and
heat treating the elongated thin tube in a temperature range of approximately 100 degrees C. to approximately 475 degrees C. for a period of from approximately 5 minutes to approximately 10 hours.
